(19) United States
(12) Patent Application Publication
(10) Pub. No.: US 2023/0392199 A1
(43) Pub. Date: Dec. 7, 2023

(54) DETECTION OF TARGET NUCLEIC ACIDS WITH PREAMPLIFICATION

(71) Applicant: Saga Diagnostics AB, Lund (SE)

(72) Inventor: Anthony Miles George, Lund (SE)

(21) Appl. No.: 18/204,688

(22) Filed: Jun. 1, 2023

Related U.S. Application Data
(60) Provisional application No. 63/348,855, filed on Jun. 3, 2022.

(51) Int. Cl. C12Q 1/6858; C12Q 1/6876

(52) U.S. Cl. CPC C12Q 1/6858; C12Q 1/6876; C12Q 1/6806

(57) ABSTRACT

The present invention provides a method for detecting low-abundance nucleic acids, including structural sequence variants and mutations. The invention provides for pre-amplification of the structural variants or mutants, which may be present in low concentration in the sample, prior to their detection using another method such as conventional PCR or digital PCR. Advantageously, the pre-amplification step, followed by PCR, results in reduction of false-negative results from the sample detection. As a result, the methods of the current invention enhance the sensitivity for sample detection, especially in detecting the presence of low-abundance targets in the biological sample.

### TECHNICAL FIELD

The present invention relates to the field of methods for detection of low abundance target nucleic acids.

### BACKGROUND

Detection of nucleic acids present in very low quantities and/or at low frequency is desirable for many applications. Detection of gene mutations is for example important for a myriad of diseases, such as cystic fibrosis, sickle cell anemia, and cancers. It is increasingly being recognized that exceptionally sensitive and specific methods for mutation detection are necessary, in particular for low-input samples such as circulating cell-free DNA (cfDNA) and analysis of single cells. Conventional methods can suffer from a constellation of issues, including a high requirement of input sample DNA quantity, high per-sample cost, complex and laborious workflows, insufficient sensitivity and/or specificity, and inability to detect low-abundance mutated sequences within a high background of normal wild-type sequence (so-called mutant allele fraction; MAF). Most detection methods rely on nucleic acid amplification using the polymerase chain-reaction (PCR) to exponentially copy regions of interest.

Digital PCR (dPCR) is a method that partitions a PCR reaction into many smaller individual reactions so that each reaction partition contains zero to only a very few target sequence molecules. The partitioning of all molecules is random and follows a Poisson distribution. The partitioning transforms the situation of an extremely low relative abundance of a rare variant sequence among an abundance of wild-type sequence, to a situation where most partitions have only wild-type sequence and some partitions have a very high relative abundance of the rare variant sequence compared to wild-type sequence. The result is a potential increase in sensitivity. However, dPCR does not always detect the presence of low abundance target nucleic acids in a biological sample. As a result, the individual suffering from diseases which require the detection of low abundance target nucleic acids in in biological samples continue to suffer.

### SUMMARY

The present invention provides methods for detecting low-abundance nucleic acids, including structural variants and mutants. The invention uses a selective pre-amplification to increase the abundance of a target of interest in a sample, even when that target is present in very low numbers. The invention solves specific problems associated with molecular detection assays in which, due to the stochastic nature of PCR, targets present in very low numbers may not be detected and in which PCR “dead volumes” are a problem. Those problems are addressed by selectively amplifying a target of interest in what is described here as a pre-amplification step. The pre-amplification step may take one of two basic forms. It may be non-exponential, e.g., may not be PCR. For example, the pre-amplification step may be incremental, which may be taken to mean that extension products from one round of pre-amplification are not copied using any reverse primer or are not substrates for copying by any reverse primer. In other preferred embodiments, the pre-amplification step exponential. In either case, the proportion of rare targets is increased as a result of the pre-amplification step.

It may be preferable to perform pre-amplification on select targets while preserving non-target material for subsequent amplification steps. Preferred embodiments use pre-amplification that is specific for a sequence of clinical significance, such as a structural variant associated with a tumor. In fact, embodiments of the invention involve identifying tumor mutations and selecting one or more structural variants.

Variant detection according to methods of the disclosure is based on selecting the target variant and conducting a pre-amplification step that is directed to the variant. In one aspect, the pre-amplification step is essentially a variant enrichment step, resulting in a sample that has a sufficient amount of a rare variant for detection. Such a pre-amplification may amplify only select targets by a limited, controlled, or known amount. For example, ten cycle of linear pre-amplification with a primer specific for a variant increases copies of that variant in the sample about tenfold. Because abundance is increased by a known amount, if sequences in the reaction mixture are later quantified (e.g., by digital PCR), the quantities of those sequences in the original sample can be determined (using the know amount by which abundance of the select variants was increased). Additionally, the pre-amplification step greatly increases the probability of detection of rare variants in a sample. The sample can then be assayed by PCR-based methods, such as digital PCR (dPCR), for the selected variant as well for any other variants potentially present in the sample. In fact, embodiments of the invention may be multiplexed using, e.g., differently labeled fluorescent hydrolysis probes to interrogate the sample for multiple variants simultaneously. Because a selected variant or variants was increased in relative abundance by the incremental pre-amplification, it will not be lost to dead volume or to stochastic effects of PCR. Because the increase due to pre-amplification is a known increase, dPCR gives a quantitative reading of the selective variant (or variants) in the sample. Thus assays of the invention are useful for quantitatively detecting rare targets in a sample and may be particularly useful for detecting cancer-specific mutations, such as in circulating tumor DNA (ctDNA) in a blood or plasma sample (e.g., in a liquid biopsy). Moreover, as discussed further herein, the incremental preamplification and the exponential amplification of dPCR can proceed in the presence of the same set of reagents (primers, dNTPs, polymerase, ions, probes . . . ) without requiring a clean-up step, a reagent change, or the addition of reagents as the assay progresses. In fact, certain embodiments discussed herein use a primer pair that functions to incrementally amplify a target under one thermocycle and exponentially amplify the target for dPCR under a different thermocycle.

In related embodiments, the invention provides for the identification and ranking of seminal or truncal variants that are among the earliest variants in oncogenesis and tumor progression. These earliest truncal variants or rearrangements are selected, prepared in a such a way to preferentially increase their copy number in the sample, and detected. The invention recognizes that early chromosomal rearrangements are superior to point mutations and are detectable according to methods of the invention. Methods of the invention allow detection of truncal variants at low concentration in a biological sample.

Methods of the invention overcome stochastic effects of PCR and the problem of dead volume by pre-amplifying target(s) of interest to increase the abundance of the target(s). The increase in abundance is controlled such that the resulting abundance is sufficient to overcome stochastic effects and the problem of dead volume but falls within the dynamic range of subsequent quantitative PCR methods, for example digital PCR. The amplification process is performed on structural sequence rearrangements that are detected by subsequent digital PCR. These processes are designed to increase the number of copies of target biomarkers and simultaneously reduce noise that often makes detection of low-prevalence sequences in a sample difficult. Methods of the invention can use an entire preamplification reaction product as input to an exponential amplification. In other words, there need not be any clean-up between the amplification steps and all nucleic acid in a sample are used as input to the subsequent process. In one embodiment, the invention is carried out using pre-amplification of variants in a sample, which may be present in low concentration in the sample, prior to an exponential amplification. Methods of the present invention enhance sensitivity in sample detection, especially in detecting the presence of low-abundance targets in a biological sample.

The present invention allows for the detection of nucleic acids in any sample comprising one or more target nucleic acids. Preferred methods of the invention comprise whole genome sequencing of tumor nucleic acid and identifying truncal variants in the tumor sequence. The truncal variants are then ranked based on a number of factors to prioritize each variant in terms of its utility as a tumor-specific biomarker timeline of tumor progression. The ranked and filtered truncal variations are selected and analyzed to determine impact on tumorigenesis. The analysis preferably is accomplished via PCR, and preferably digital PCR. The invention allows the identification of early truncal variants in a biological sample as biomarkers for analysis. In one aspect, pre-amplification a sample preparation to preferentially increase the number of copies of the selected biomarkers is performed on the nucleic acids in the sample to increase copy number of the target nucleic acid (e.g., variants) in the sample. The sample is then aliquoted into a plurality of subsamples and PCR/dPCR is conducted on the subsamples. The target nucleic acids are then analyzed and/or detected in the sample.

Preferred methods of the invention include a pre-amplification step. By way of example, the pre-amplification may be an asymmetric incremental amplification or symmetric exponential amplification, or a combination of the two. Incremental pre-amplification may include the use of only a single primer per target of interest, or may use primer pairs, wherein only one primer is active in the pre-amplification. In one example, the active primer is active due to a difference in the melting temperature (Tm) and annealing temperatures of the provided primers.

Asymmetrical incremental amplification may comprise providing a pair of primers capable of amplification of a target nucleic acid. For example, the pair of primers may comprise a primer-H and a primer-L. The melting temperature of primer-H may be about 10° C. to about 22° C. higher than the melting temperature of primer-L. Primer-L may comprise a sequence complementary to a fragment of the elongation product of primer-H. The melting temperature of primer-H may generally be about 10° C. higher than the melting temperature of primer-L. For example, the melting temperature of primer-H may be about 10° C. higher than the melting temperature of primer-L, about 12° C. higher than the melting temperature of primer-L, or about 16° C. higher than the melting temperature of primer-L.

The sample preparation reaction (e.g., the incremental amplification and/or an exponential amplification) may also include nucleic acid polymerases having polymerase activity, additional primer or primers, and reagents typically employed in standard PCR.

For example, asymmetric incremental amplification may include a set of primers, wherein at least one primer is specifically capable of amplification of only one strand of the target nucleic acid sequence. With the inclusion of the primers, the sample preparation reaction in a solution including a nucleic acid polymerase having polymerase activity may then be performed. The sample preparation reactions may also include nucleic acid polymerase having polymerase activity, primer(s), and reagents typically employed in standard PCR.

Symmetric exponential amplification may also be used in methods of the invention. Symmetric exponential amplification may include the provision of a set of primers specifically capable of amplification of the target nucleic acid sequence. A nucleic acid polymerase having polymerase activity at an elongation temperature is then provided to the sample. A sample preparation reaction is then prepared comprising a part of the sample, the set of primers, the nucleic acid polymerase, and reagents typically employed in standard PCR; and performing the sample preparation reaction.

Asymmetrical incremental amplification may be followed by, or combined with, symmetrical exponential amplification. Symmetrical exponential amplification may be followed by asymmetrical incremental amplification. Asymmetric incremental amplification and symmetric exponential amplification may also be performed in the same reaction. Advantageously, this allows for asymmetric incremental amplification and symmetric exponential amplification to be performed using the same set of primers.

Asymmetric incremental amplification may be activated at a higher temperature as compared to symmetric exponential amplification. For example, the primer or primers for asymmetric incremental amplification may have a different annealing temperature as compared to the annealing temperature of primer or primers for symmetric exponential amplification. Furthermore, the thermal cycling conditions for asymmetric incremental amplification may have a different annealing temperature as compared to the annealing temperature for symmetric exponential amplification

The invention further includes the step of conducting a plurality of PCR reactions on the sample, which has undergone pre-amplification. The PCR step after pre-amplification may include a pair of primers capable of specific amplification of a target nucleic acid and a nucleic acid polymerase having a polymerase activity at an elongation temperature. Each PCR reaction comprises a part of the sample, the set of primers, the nucleic acid polymerase, PCR reagents. The PCR step may comprise digital PCR (dPCR).

The amplicon produced during a pre-amplification step such as a variant enrichment sample preparation reaction step may be used as direct sample input for the PCR. That is, after the pre-amplification, reagents for PCR may be added to the reaction mixture from the pre-amplification. For example, if the pre-amplification is performed in tube, after pre-amplification, the tube can be topped-up (without clean-up) to add any additional reagents useful in PCR. In some embodiments, primers used in the invention are a part of a plurality of pairs of primers capable of amplification of different target nucleic acid sequences. Pre-amplification can be performed under a first temperature control then, after optionally adding any further reagents to the tube and optionally without any clean-up, PCR may be performed under a second temperature control. Methods of the invention may be performed without any cleanup steps, which could lead to loss of analyte material.

Methods of the invention may further include the use of a plurality of pairs of primers capable of amplification of different target nucleic acid sequences. In certain embodiments, methods of the invention may include the use of multiplex PCR.

Methods of the invention may provide that the one or more targeted nucleic acid molecules are associated with a variant sequence, wherein the variant sequence may be a variant of a wildtype nucleic acid sequence. The variant sequence may be selected from the group consisting of single nucleotide variants (SNVs), insertions and deletions (indels), duplications, copy-number variants (CNVs), inversions, fusions, and translocations. The methods may also be used to analyze the cfDNA samples from a cancer patient, optionally including mutant or structural variants from cfDNA.

Certain methods of invention include a sample preparation to preferentially increase the number of copies of the target nucleic acid in the sample prior to PCR. As a result of the targeted sample preparation, an increased number of copies of the target are available for the PCR reactions. An advantage of the sample preparation is that it reduces stochastic bias in samples with low concentration of target. Furthermore, an advantage is that it reduces the problem of dead volume as the chance that a target will only be present in the dead volume is dramatically reduced.

The invention also provides a reference assay for detection of structural variants or mutants of patient's cfDNA. In particular, the invention provides methods for detecting the wildtype cfDNA as a baseline. Accordingly, aspects of the invention provide an assay comprising the use of a pair of primers and probe, to detect and/or quantify a wildtype (reference) sequence of cfDNA.

The invention further provides an assay to calculate variant allele fraction (VAF) for the target nucleic acids in the sample. Embodiments of methods of the invention include calculation of efficiency of the replication, and thus, quantifying the amount of target nucleic acid present in the sample. The invention further provides for two positive control reactions during the pre-amplification. The first positive control comprises positive control DNA in the assay, so that pre-amplification occurs. The second positive control comprises an equal amount of positive control DNA, without the primers. The first positive control and the second positive control are technical replicates with the exception that first positive control receives the primer(s). Subsequently, the concentration of the replicate with primers is divided by the replicate with no primers to estimate the efficiency of assay. The efficiency calculated can be applied to the measurements performed on the actual samples. As a result, methods of the invention may be used to quantify the amount of target nucleic acid in the sample.

### BRIEF DESCRIPTION OF THE DRAWINGS

FIG. 1 diagrams methods of the invention.

FIG. 2 gives workflows for sample acquisition, bioinformatics and sample processing.

### DETAILED DESCRIPTION

The present invention provides methods for detecting low-abundance nucleic acids, including structural variants and mutants. In one aspect, the invention provides sample preparation steps to preferentially increase the number of copies of structural variants or mutants, which may be present in low concentration in the sample prior to digital PCR (dPCR). Sample preparation steps function to incrementally amplify variants of interest, followed by dPCR, results in reduction of false-negative results from the sample. As a result, methods of the current invention enhance the sensitivity for sample detection, especially in detecting the presence of low-abundance targets in the biological sample.

As described above, methods of the invention are especially useful for detection of low-abundance target nucleic acids in a sample. A target nucleic acid is any nucleic acid sequence the presence of which is desirable to detect. The target nucleic acid or variant may for example be a nucleic acid sequence associated with a clinical condition. In particular, a variant associated with early tumorigenesis (a truncal variant) is identified and ranked according to a number of factors. The selected truncal variants are amplified for detection and characterization. The characterization may include a report on the clinical status of the tumor and patient (e.g., whether it is primary, recurrent, evidence of minimal residual disease).

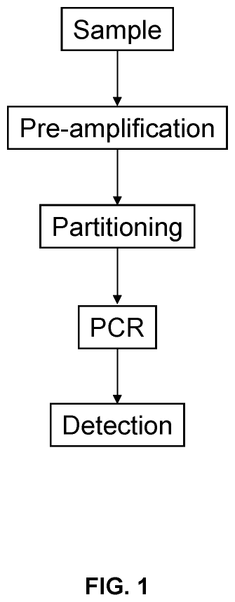

As described in FIG. 1, the present invention provides, in one non-limiting example, a method including the steps of: (i) providing a sample comprising one or more target nucleic acids, (ii) performing pre-amplification to selectively increase copy number of the target nucleic acid in the sample, (iii) aliquoting the sample into a plurality of subsamples, each in its own partition; (iv) conducting polymerase chain reaction (PCR) on the subsamples, and (v) detecting the targe nucleic acids. Preferably, the PCR step (iv) is digital PCR, e.g., with fluorescent probes such as hydrolysis probes that show the presence of amplicon being produced in each partition.

Methods of the present invention are useful for detecting whether a variant sequence is present in a sample, which may comprise a mixture of target nucleic acids wherein only a portion of the target nucleic acids may comprise the variant sequence. In particular, the methods are useful for detecting the presence of a variant sequence in a sample comprising target nucleic acids of which only a minor fraction may potentially comprise the variant sequence.

The present invention also provides methods for detection of the presence of a target nucleic acid sequence in a sample. The methods may be useful for detecting whether a target nucleic acid sequence is present in a sample. The sample may comprise a mixture of template nucleic acids potentially comprising the target nucleic acid sequence. In particular, the methods are useful for detecting the presence of a target nucleic acid sequence in a sample, which potentially may comprise only a very low level of said target nucleic acid sequence.

The target nucleic acid sequence may be any target nucleic acid sequence, which is desirable to detect. For example, the presence of the target nucleic acid sequence may be associated with a clinical condition.

According to the methods of the invention, a method of detecting the presence of a target nucleic acid sequence, wherein the method includes providing a sample comprising one or more target nucleic acids, and performing pre-amplification to increase copy number of the target nucleic acid in the sample. Subsequently, the pre-amplified sample is aliquoted into a plurality of sub samples, conducting a PCR (preferably dPCR) on the subsamples; and detecting the target nucleic acids.

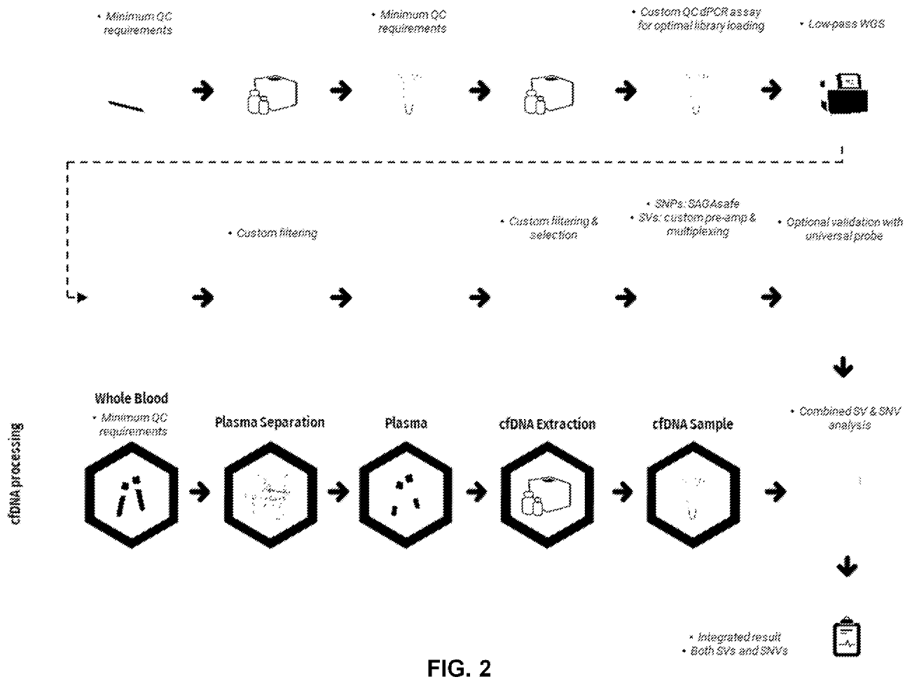

FIG. 2 provides an exemplary workflow showing sample acquisition, bioinformatics workflow and sample processing. This exemplary workflow shows low pass whole genome sequencing of the tumor as a the first step, followed by filtering and selection of the target, which may be a structural variant or a mutation (or the combination thereof).

Methods of the invention comprise the use of an incremental nucleic acid amplification step as a targeted pre-amplification, i.e., to increase relative abundance of a target within a sample. The use of targeted pre-amplification increases the number of copies of the target nucleic acid in the sample. The pre-amplification could be symmetrical or asymmetrical, or a combination of the two. In an asymmetrical or incremental amplification, a single strand of nucleic acids is linearly copied, preferentially without copying other strands among the sample. Some embodiments perform incremental amplification by providing only one primer that copies a target (instead of the forward- and reverse-primer pair of PCR). Certain embodiments discussed herein perform the asymmetrical amplification using a primer-H and a primer-L. See also U.S. Pat. No. 11,066,707 and U.S. Pub. 2022/0056533 A1, both incorporated by reference.

The methods of the invention have a very low limit of detection. This enables a detection of target nucleic acid sequence potentially present at very low levels, and/or detection of the presence of variant sequences potentially present at very low levels in mixtures comprising other target nucleic acid sequences.

### Sample and Target Nucleic Acids

The sample may be any sample in which it is desirable to detect, whether said variant sequence is present. For example, if the variant sequence is indicative of a clinical condition, the sample may be a sample from an individual at risk of acquiring said clinical condition. The variant sequence may differ from the wild-type sequence by substitution(s), deletion(s) and/or insertions(s).

The methods of the invention further provide that the one or more targeted nucleic acid molecules are associated with a variant sequence, wherein the variant sequence may be a variant of a wildtype nucleic acid sequence. The variant sequence is selected from the group consisting of single nucleotide variants (SNVs), insertions and deletions (indels), duplications, copy-number variants (CNVs), inversions, and translocations. The methods of the invention may also be used to analyze the cfDNA samples from a cancer patient, optionally including mutant or structural variants from cfDNA.

For polymorphisms or small indels (typically <about 50 bases), it may be preferred that both the wild-type sequence and the variant sequence be amplified in a polymerase reaction by the pair of primers specifically capable of amplification of the target nucleic acid sequence. It may be preferred that the wild-type sequence and the variant sequence does not differ too much from each other in length. For structural rearrangements (typically at least about 50 bases), the variant is amplified by an incremental step prior to digital PCR and assays also, in parallel, include a reference assay targeting a stable region of the genome to quantify the signal. Structural variants are described in Mahmoud, 2019, Structural variant calling: the long and the short of it, Genome Biology 20:a246, incorporated by reference.

The sample may be any biological sample, including a bodily fluid sample comprises bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, or vomit.

### Amplification and Pre-Amplification

The methods of the current invention involve the use of amplification. Amplification of a nucleic acid is the generation of copies of said nucleic acid.

The methods of the invention also include a pre-amplification step. The pre-amplification may be described as a variant enrichment sample preparation reaction step. Such a pre-amplification may amplify only select targets by a limited, controlled, or known amount. For example, ten cycle of linear pre-amplification with a primer specific for a variant would increase abundance of copies of that variant in the sample about tenfold. Because the abundance is increased by a known amount, if sequences in the reaction mixture are later quantified (e.g., by digital PCR), the quantities of those sequences in the original sample can be determined (using the know amount by which abundance of the select variants was increased). Additionally, the pre-amplification greatly increases the probability of detection of those variants, which aids in detection of very rare sequences in a sample. That is why it may be preferable to have the pre-amplification specifically increase the abundance of copies of the selected variant of the sample by known amounts. According to the invention, the pre-amplification step is a sample preparation to preferentially increase the number of copies of the selected targets through asymmetric incremental amplification or symmetric exponential amplification or a combination of the two. An incremental pre-amplification could be expected to proceed without more than linear copy amplification, compared to the exponential production of amplicons in PCR. The incremental amplification may proceed using un-paired primers (e.g., single primers) that get extended through, and copy, a target of interest. In some embodiments, an incremental pre-amplification comprises the use of at least a couple of primers, wherein only one primer is active in the pre-amplification. The said active primer may be active due to a difference in the melting temperature (Tm) and annealing temperatures of the primers. The use of these primers for asymmetrical incremental amplification is disclosed in U.S. Pat. No. 11,066,707, which is hereby incorporated by reference in its entirety.

Methods of the invention provides that the nucleic acid polymerase enzymes may have different elongation temperatures. The elongation temperature is a temperature allowing enzymatic activity of the nucleic acid polymerase following primer annealing. Typically, a nucleic acid polymerase has activity over a temperature range, and thus the elongation temperature may be any temperature within that range. Most nucleic acid polymerases have a temperature optimum but retain activity at other temperatures than the temperature optimum. In such cases, the elongation temperature may be any temperature where the primer anneals and the nucleic acid polymerase has activity even if the temperature is not the optimum temperature. At the elongation temperature of a nucleic acid polymerase, the enzyme is capable of catalyzing synthesis a new nucleic acid strand complementary to the template strand at the elongation temperature. The invention provides that the elongation temperature is near the melting temperature of the primer-H. Thus, a nucleic acid polymerase may be chosen, which has polymerase activity at a temperature near the melting temperature of primer-H and/or the primer-H may be designed to have a melting temperature near the elongation temperature.

The amplicons or product produced during a pre-amplification step such as a variant enrichment sample preparation reaction step may be used as direct sample input for the PCR. That is, after the pre-amplification, reagents for PCR may be added to the reaction mixture from the pre-amplification. For example, if the pre-amplification is performed in a tube, after pre-amplification, the tube can be topped-up (without clean-up) to add any additional reagents useful in PCR. In some embodiments, primers used in the invention are a part of a plurality of pairs of primers capable of amplification of different target nucleic acid sequences. Pre-amplification can be performed under a first temperature control then, after optionally adding any further reagents to the tube and optionally without any clean-up, PCR may be performed under a second temperature control. Methods of the invention may be performed without any cleanup steps, which could lead to loss of analyte material.

Methods of the invention involve the use of PCR reagents for the sample preparation reactions. The sample preparation reaction should comprise at least part of the sample, the set of primers, and sufficient PCR reagents to allow a polymerase reaction. Methods and reagents useful for performing a PCR reaction are well known to the skilled person. For example, the PCR reaction may comprise any of the nucleic acid polymerase and PCR reagents, described herein below in the section “PCR reagents”. Depending on the mode of detecting whether the sample preparation product comprises the variant sequence, the sample preparation reaction may also comprise detection reagents. PCR reagents are reagents which are added to a PCR in addition to nucleic acid polymerase, sample and set of primers. The PCR reagents at least comprise nucleotides. In additional the PCR reagents may comprise other compounds such as salt(s) and buffer(s).

For most purposes the PCR reagents comprise nucleotides. Thus, the PCR reagents may comprise deoxynucleoside triphosphates (dNTPs), in particular all of the four naturally-occurring deoxynucleoside triphosphates (dNTPs).

The PCR reagents frequently comprise deoxyribonucleoside triphosphate molecules, including dATP, dCTP, dGTP, dTTP. In some cases, dUTP is added.

The PCR reagents may also comprise compounds useful in assisting the activity of the nucleic acid polymerase. Thus, the PCR reagent may comprise a divalent cation, e.g., magnesium ions. Said magnesium ions may be added on the form of e.g. magnesium chloride or magnesium acetate (MgCl2) or magnesium sulfate is used.

The PCR reagents may also comprise one or more of the following:
non-specific blocking agents such as BSA or gelatin from bovine skin, betalactoglobulin, casein, dry milk, or other common blocking agents,
non-specific background/blocking nucleic acids (e.g., salmon sperm DNA),
biopreservatives (e.g. sodium azide),
PCR enhancers (e.g. Betaine, Trehalose, etc.),
inhibitors (e.g. RNAse inhibitors).

The PCR reagent a may also contain other additives, e.g., dimethyl sulfoxide (DMSO), glycerol, betaine (mono)hydrate (N,N,N-trimethylglycine=[caroxy-methyl]trimethylammonium), trehalose, 7-Deaza-2′-deoxyguanosine triphosphate (dC7GTP or 7-deaza-2′-dGTP), formamide (methanamide), tettrmethylammonium chloride (TMAC), other tetraalkylammonium derivaties (e.g., tetraethyammonium chloride (TEA-Cl) and tetrapropylammonium chloride (TPrA-Cl), non-ionic detergent (e.g., Triton X-100, Tween 20, Nonidet P-40 (NP-40)), or PREXCEL-Q.

The PCR reagents may comprise a buffering agent.

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%, or 1.0%. Common biosurfactants include non-ionic surfactants such as Pluronic F-68, Tetronics, Zonyl FSN. Pluronic F-68 can be present at a concentration of about 0.5% w/v.

A wide range of common, commercial PCR buffers from varied vendors can be substituted for the buffered solution.

The methods of the invention also involves use of a nucleic acid polymerase. Said nucleic acid polymerase may be any nucleic acid polymerase, such as a DNA polymerase. The nucleic acid polymerase should have activity at the elongation temperature.

The nucleic acid polymerase may be a DNA polymerase with 5′ to 3′ exonuclease activity. This may in particular be the case in the methods of the invention, wherein the methods or kits involves use of a detection probe, such as a Taqman detection probe.

Any DNA polymerase, e.g., a DNA polymerase with 5′ to 3′ exonuclease activity that catalyzes primer extension can be used. For example, a thermostable DNA polymerase can be used. Preferably, the nucleic acid polymerase is a Taq polymerase.

The methods of the invention include a sample preparation reaction step, which could be either asymmetric incremental amplification or symmetrical exponential amplification or a combination of the two. Asymmetric incremental amplification may proceed using unpaired, e.g., single primers (even if multiple primers are used, each may be “single” in the sense of not being paired with a reverse primer that anneals to the extension product of the single primer). In some embodiments, asymmetric incremental pre-amplification may include the use of at least a couple of primers, wherein only one primer is active in the sample preparation reaction. For example, the said active primer is active due to a difference in the melting temperature (Tm) and annealing temperatures of the provided primers.

More preferably, the step of asymmetrical incremental amplification includes: (i) providing a pair of primers capable of amplification of a target nucleic acid, wherein the pair of primers comprises a primer-H and a primer-L. According to the methods of the invention, the melting temperature of primer-H may be about 10° C. to about 22° C. higher than the melting temperature of primer-L, and primer-L comprises a sequence complementary to a fragment of the elongation product of primer-H, and the sample preparation reaction is performed. The sample preparation reactions also include nucleic acid polymerase having polymerase activity, primer(s), and PCR reagents.

In the embodiments with primer-H and primer-L, the incremental amplification will proceed using an annealing temperature at which primer-H (but not primer-L) anneals. During that incremental amplification, primer-H will function as a “single” primer (despite the presence of primer-L). Primer-L will not anneal to anything to any meaningful extent because the reaction mixture is not brought down to the annealing temperature of primer-L. The incremental preamplification may be run for a predetermined number of cycles (e.g., one, or five, etc.) or for a fixed amount of time, or until the sample exhibits a results (change in optical density, cleavage of a fluorescent probe, etc.). After the incremental amplification, then the reaction mixture may be subject to exponential amplification. For the exponential amplification, at the annealing step, the temperature is brought down to the annealing temperature of primer-L, which promotes annealing of both primer-H and primer-L.

It is important to note that preceding paragraphs describes the function of primer-H and primer-L as those primers may function among many other primers. For example, tens, hundreds, or thousands, or more loci can be probed in parallel using a corresponding number of primer pairs. For an example, we'll say that 24 loci are being probed in parallel (but that number 24 is arbitrary, and could just as easily be 1, or 2, or 3, or 6, or 17, or 96, or 99, or 384, or 1,000, or 1,536, or an integer multiple of any of those numbers, etc.) An assay of the invention may use a primer pair for each loci, e.g., may use 24 primer pairs. Any one or any number of those primer pairs may fit the description of primer-H and primer-L. However, and this is important, it may be desirable to preamplify only certain loci, so any number of the primer pairs may include forward and reverse primer that each have an annealing temperature essentially the same as for a primer-L.

In other embodiments, the pre-amplification proceed with unpaired “single” primers (e.g., that operate at the primer-H annealing temperature. After that, the reaction mixture (original sample, plus added reagents, plus preamplifcation product) may be subject to conditions for exponential amplification. The exponential amplification may use primer pairs, any one of which may “match” all or part of the single primer, or than anneal to targets within the length of the extension product of the unpaired single primers. Those reactions may proceed at different temperatures, or the paired primers may not be made available until after the incremental preamplification. For example, the paired primers may be added (e.g., by microfluidic handling) or released from confinement or attachment (e.g., by chemical, temperature, or photo lysis of a hydrogel bead).

A feature in common among the embodiments is that they allow all reaction reagents to be added at the beginning and each step can proceed regardless of the presence of other reaction reagents. For example, primer-H/primer-L embodiments operate first at a higher temperature at which primer-L is essentially unused. In another example, paired primers could be sequestered from use in a hydrogel bead until release by a change such as in pH or redox or photocleavage. A key feature is that where a final readout is digital PCR in aqueous partitions, an incremental preamplification may be performed in those partitions or prior to partitioning in “bulk phase”, without the requirement of changing or adding reagents when progressing through the steps of preamplification, partitioning, dPCR.

In general, embodiments may include denaturing DNA to single-stranded molecules, ii) incubating the molecules with primer-H, primer-L and reagents for amplification plus dPCR readout at a high annealing temperature allowing annealing of primer-H, but not of primer-L, optionally incubating at an elongation temperature, (optionally cycling melt, anneal, and elongate steps), partitioning into partitions, performing a polymerase chain reaction (PCR) using a low annealing temperature allowing annealing of both primer-H and primer-L, and detecting whether the partitions comprise amplicons comprising a target or variant of interest.

The invention provides that the melting temperature of primer-H may be about 10° C. higher than the melting temperature of primer-L. Preferably, the melting temperature of primer-H may be about 10° C. higher than the melting temperature of primer-L. More preferably, the melting temperature of primer-H is about 12° C. higher than the melting temperature of primer-L. More preferably, the melting temperature of primer-H is about 16° C. higher than the melting temperature of primer-L.

In methods of the invention, the asymmetrical incremental amplification comprises: providing a pair of primers capable of amplification of a target nucleic acid, wherein the pair of primers comprises a primer-H and a primer-L, wherein the melting temperature of primer-H is about 10° C. to about 22° C. higher than the melting temperature of primer-L, and wherein primer-L comprises a sequence complementary to a fragment of the elongation product of primer-H; providing a nucleic acid polymerase having polymerase activity at an elongation temperature; and preparing sample preparation reactions, each comprising a part of the sample, the set of primers, the nucleic acid polymerase, and PCR reagents. The melting temperature of primer-H may be about 10° C. higher than the melting temperature of primer-L. Preferably, the melting temperature of primer-H is about 12° C. higher than the melting temperature of primer-L. More preferably, the melting temperature of primer-H is about 16° C. higher than the melting temperature of primer-L.

According to the invention, the asymmetric incremental amplification may also include a set of primers, wherein at least one primer is specifically capable of amplification of only one strand of the target nucleic acid sequence, and performing the sample preparation reaction in a solution including a nucleic acid polymerase having polymerase activity, and the sample preparation reaction is performed. The sample preparation reactions also include nucleic acid polymerase having polymerase activity, primer(s), and PCR reagents.

The invention may further include a step of symmetric exponential amplification, wherein the symmetric exponential amplification include the steps of (i) providing a set of primers specifically capable of amplification of the target nucleic acid sequence; (ii) providing a nucleic acid polymerase having polymerase activity at an elongation temperature; (iii) preparing sample preparation reactions each comprising a part of the sample, the set of primers, the nucleic acid polymerase, and PCR reagents; and performing the sample preparation reaction.

The methods of the current invention provide that asymmetrical incremental amplification is followed by symmetrical exponential amplification. The steps involved in asymmetrical incremental amplification and symmetrical exponential amplification are outlined above.

The methods of the current invention further provide that symmetrical exponential amplification may be combined with, follows, or is followed by asymmetrical incremental amplification. The steps involved in asymmetrical incremental amplification and symmetrical exponential amplification are outlined above.

The methods of the current invention also provide that asymmetric incremental amplification and said symmetric exponential amplification may be performed in the same reaction volume. According to the methods of the invention, asymmetric incremental amplification and said symmetric exponential amplification may be performed using the same set of primers.

The method of the invention also provides that asymmetric incremental amplification may be activated at a higher temperature as compared to symmetric exponential amplification. For example, a thermocycler may be programmed to go down only to the higher annealing temperature for the incremental amplification but to go to a lower annealing temperature for exponential amplification.

The invention further includes the step of conducting a plurality of PCR reactions on the sample, which has undergone the sample preparation reaction, wherein the step includes a pair of primers capable of specific amplification of a target nucleic acid, a nucleic acid polymerase having a polymerase activity at an elongation temperature, preparing PCR reactions, wherein each PCR reaction comprises a part of the sample, the set of primers, the nucleic acid polymerase, PCR reagents; and performing symmetrical exponential amplification. The invention provides that these PCR reactions could be conventional PCR. Preferably, the PCR is digital PCR (dPCR). According to the methods of the invention, the sample including the products following the sample preparation reaction step may be used as direct sample input for the PCR. Optionally, the pair of primers used in the invention are a part of a plurality of pairs of primers capable of amplification of different target nucleic acid sequences.

The methods of the invention may further include the use of a plurality of pairs of primers capable of amplification of different target nucleic acid sequences. Preferably, the methods of the invention include the use of multiplex PCR.

Methods of the invention may use quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), digital PCR (dPCR), droplet digital PCR (ddPCR), bridge PCR, picotiter PCR, and emulsion PCR.

Primer-H and Primer-L The methods of the invention also include the use of several primers including primers named as primer-H and primer-L. In context of the methods of the invention, primer-H is a primer having a high melting temperature, whereas primer-L is a primer having a low melting temperature. The melting temperature of a primer is the temperature at which 50% of the primer forms a stable double helix with its complementary sequence and the other 50% is separated to single strand molecules. The melting temperature may also be referred to as Tm or Tm. Preferably, the Tm as used herein is calculated using a nearest-neighbor method based on the method described in Breslauer, 1986, Predicting DNA duplex stability from the base sequence, PNAS 83:3746-50 (incorporated by reference) using a salt concentration parameter of 50 mM and primer concentration of 900 nM. For example, the method is implemented by the software “Multiple Primer Analyzer” from Life Technologies/Thermo Fisher Scientific Inc.

The methods of the invention involves use of a set of primers comprising a primer-H and a primer-L, wherein the melting temperature of primer-H is about 10° C. to about 22° C., preferably at least 10° C., more preferably at least 15° C. higher than the melting temperature of primer-L, and wherein primer-L contains a sequence complementary to the elongation product of primer-H.

The primer-H is preferably designed as a primer for amplification of the target sequence or the sequence complementary to the target sequence. Thus, the primer-H is preferably capable of annealing to either the target nucleic acid sequence or to the sequence complementary to the target nucleic acid sequence. For example, primer-H may be capable of annealing to the complementary strand of the target nucleic acid sequence at the 5′-end or close to the 5′-end of the target nucleic acid sequence, or the primer-H may be capable of annealing to the target nucleic acid sequence at the 3′-end or close to the 3′-end of the target nucleic acid sequence. Thus, the primer-H may comprise a sequence identical to the 5′-end of the target nucleic acid sequence. The primer-H may even consist of a sequence identical to the 5′-end of the target nucleic acid sequence. The primer-H may also comprise a sequence identical to the target nucleic acid sequence. Thus, the primer-H may comprise a sequence complementary to the 3′-end of the target nucleic acid sequence. The primer-H may even consist of a sequence complementary to the 3′-end of the target nucleic acid sequence.

Similarly, the primer-L is preferably designed as a primer for amplification of the target sequence or the sequence complementary to the target sequence. If the primer-H is designed for amplification of the target sequence, the primer-L is preferably designed for amplification of the sequence complementary to the target sequence and vice versa. Thus, the primer-L is preferably capable of annealing to either the target nucleic acid sequence or to the sequence complementary to the target nucleic acid sequence. If primer-H is capable of annealing to the target nucleic acid sequence, then primer-L is preferably capable of annealing to the sequence complementary to the target nucleic acid sequence and vice versa. For example, primer-L may be capable of annealing to the complementary strand of the target nucleic acid sequence at the 5′-end or close to the 5′-end of the target nucleic acid sequence, or the primer-L may be capable of annealing to the target nucleic acid sequence at the 3′-end or close to the 3′-end of the target nucleic acid sequence. Thus, the primer-L may comprise a sequence identical to the 5′-end of the target nucleic acid sequence. The primer-L may even consist of a sequence identical to the 5′-end of the target nucleic acid sequence. The primer-L may also comprise a sequence identical to the target nucleic acid sequence. Thus, the primer-L may comprise a sequence complementary to the 3′-end of the target nucleic acid sequence. The primer-L may even consist of a sequence complementary to the 3′-end of the target nucleic acid sequence.

The primer-H may have a nucleotide sequence identical to the sequence at the 5′-end of the target nucleic acid sequence and the primer-L comprises or consists of a sequence identical to the complementary sequence of the 3′-end of the target nucleic acid sequence.

The primer-L may have a nucleotide sequence identical to the sequence at the 5′-end of the target nucleic acid sequence and the primer-H comprises or consists of a sequence identical to the complementary sequence of the 3′-end of the target nucleic acid sequence.

The primer-H and the primer-L are designed to have the melting temperatures as indicated herein. The skilled person will be capable of designed primer-H and primer-L to have the desired melting temperature by adjusting the sequence of the primers, the length of the primers and optionally by incorporating nucleotide analogues as described herein above in the section “Set of primers”.

The primer-H is designed so that the primer-H has an annealing temperature which is significantly higher than the annealing temperature of primer-L, for example at least 10° C. higher. Thus, the melting temperature of the primer-H may be at least 12° C. higher, for example at least 15° C. higher, preferably at least 14° C. higher, even more preferably at least 16° C. higher, yet more preferably 18° C. higher, such as at least 20° C. higher, for example in the range of 15 to 50° C., such as in the range of 15 to 40° C., for example in the range of 15 to 25° C. higher than the melting temperature of the primer-L.

In general, it may be preferred that the melting temperature of primer-H is as high as possible, but not higher than the highest functional elongation temperature of at least one nucleic acid polymerase. Said elongation temperature does not need to be the optimum temperature for said nucleic acid polymerase, but it is preferred that at least one nucleic acid polymerase has activity at the melting temperature primer-H. Thus, the melting temperature of the primer-H may approach or may even exceed 80° C.

Since it is also preferred that the melting temperature of primer-L is sufficiently high to ensure specific annealing of primer-L to the target nucleic acid sequence/the complementary sequence of the target nucleic acid sequence, and the melting temperature of primer-H should be significantly higher than the melting temperature of primer-H, then frequently, the melting temperature of primer-H is at least 60° C. The melting temperature of primer-H may also frequently be at least 70° C. The melting temperature of primer-H may for example be in the range of 60 to 90° C., for example in the range of 60 to 85° C., such as in the range of 70 to 85° C., for example in the range of 70 to 80° C.

The melting temperature of primer-L is preferably sufficiently high to ensure specific annealing of primer-L to the target nucleic acid sequence/the complementary sequence of the target nucleic acid sequence, but also significantly lower than the melting temperature of primer-H. Frequently, the melting temperature of the primer-L is in the range of 30 to 55° C., such as in the range of 35 to 55° C., preferably in the range of 40 to 50° C.

The methods of the invention may also include the use of a set primers or a plurality of primers. A set of primers or a plurality of primers contain two or more different primers. A set of primers contains at least a pair of primers specifically capable of amplification of a target nucleic acid. Furthermore, a set of primers according to the invention contains at least a primer-H and a primer-L. Thus, wherein the set of primers contains only two different primers, then set of primers contains a primer-H and a primer-L, wherein the primer-H and primer-L are capable of amplification of a target nucleic acid.

### Detection

The methods of the invention in general comprise a step of detecting, whether the sample comprises the variant sequence and/or the target nucleic acid sequence. Said detection may be accomplished in any suitable manner known to the skilled person. For example numerous useful detection methods are known in the prior art, which can be employed with the methods of the invention.

The step of detection may include the presence of a detection reagent in the PCR reactions. Said detection reagent may be any detectable reagent, for example it may be a compound comprising a detectable label, wherein said detectable label for example may be a dye, a radioactive activity, a fluorophore, a heavy metal or any other detectable label.

Frequently the detection reagent comprises a fluorescent compound.

The detection reagent may include detection probes. Detection probes may include nucleotide oligomers or polymers, which optionally may comprise nucleotide analogues. Frequently, the detection probe may be a DNA oligomer. Typically, the detection probe is linked to a detectable label, for example by a covalent bond. The detectable label may be any of the aforementioned detectable labels, but frequently it is a fluorophore.

The detection probe is in general capable of specifically binding the target nucleic acid sequence. For example, the detection probe may be capable of specifically binding the target nucleic acid comprising the variant sequence. Thus, the detection probe may be capable of annealing to the target nucleic acid sequence or to the sequence complementary to the target nucleic acid sequence. Thus, the detection probe may comprise a sequence identical to a fragment of the target nucleic acid sequence or the sequence complementary to the target nucleic acid sequence. It is generally preferred that the detection probe comprises a sequence different to the sequence of any of the primers of the set of primers.

### Quantification

As explained above, the invention comprises providing a sample comprising one or more target nucleic acids; performing sample preparation reaction to increase copy number of the target nucleic acid in the sample; aliquoting the sample into a plurality of subsamples; conducting polymerase chain reaction (PCR) on the subsamples; and detecting the target nucleic acids. As a result of the targeted pre-amplification, an increased number of copies of the target is available for the PCR reactions. An advantage of the pre-amplification is that it significantly reduces or eliminates the instance of false-negatives, which is often an issue in samples with low concentration of target, by reducing the problem of dead volumes and stochastic sampling error.

The invention also provides a reference assay for detection of structural variants or mutants of patient's cfDNA. In particular, the invention provides a method for detecting the wildtype cfDNA. Advantageously, if no mutant or structural variant targets are detected, the invention would allow for the detection of cfDNA. Accordingly, the invention provides an assay, comprising the use of a pair of primers and probe, to detect and/or quantify a wildtype (reference) sequence of cfDNA. The invention further provides an assay to calculate variant allele fraction (VAF) for the target nucleic acids in the sample. The target nucleic acid may be a variant of the wild-type nucleic acid sequence in the sample.

The methods of the invention may further provide the use of publicly available information to determine the regions of the genome that are stable for amplification and design the assay accordingly. As an example, a person of ordinary skill in the art may determine that chromosome 2, band 13 (2p13) is stable, and consequently, less susceptible to changes in copy numbers. The invention further provides that this information may be used to design the assays, especially to minimize the variations in copy numbers in the sample.

Advantageously, the invention provides a method for quantification of the amount of the target nucleic acid present in the sample. The replication assays have variability in the pre-amplification step. In particular, the efficiency of pre-amplification is less than 100%. Thus, a correction factor is applied to back-calculate original sample concentrations. As an example of this issue, the pre-amplification step may result in 50-fold amplification in one instance but can only generate 30-fold amplification in another instance, resulting in 50-fold or 30-fold copies of the target sample respectively. Moreover, denaturation state of the sample also impacts how many copies are measured by dPCR. As an example, identical samples can have a two-fold difference in dPCR concentrations depending on denaturation state (one intact double-strand DNA molecule in one compartment is measured as one copy, two single-strand DNA molecules in two compartments is measured as two copies). The methods of the invention also provide for two positive control reactions during the pre-amplification. The first positive control comprises positive control DNA and the assay, so that pre-amplification occurs. The second positive control comprises an equal amount of positive control DNA, without the primers. The first positive control and the second positive control are technical replicates with the exception that first positive control receives the primer(s).

Subsequently, the concentration of the replicate with primers is divided by the replicate with no primers to estimate the efficiency of assay. The efficiency calculated can be applied to the measurements performed on the actual samples. In effect, the invention provides a method to quantify the amount of target nucleic acid in the sample.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method comprising:
identifying sequences under-represented in a sample;
performing a pre-amplification reaction to increase abundance of copies of the identified sequences within the sample;
performing an exponential amplification reaction on nucleic acids from the sample; and
analyzing the sample for the identified sequences.

2. The method of claim 1, wherein the pre-amplification creates an exponential or linear increase in the abundance.

3. The method of claim 2, wherein the identified sequences include structural rearrangements specific to a tumor.

4. The method of claim 3, wherein the analyzing step detects minimal residual disease (MRD) after treatment.

5. The method of claim 1, wherein the sample comprises blood or plasma and the pre-amplification amplifies cell-free DNA.

6. The method of claim 3, wherein the analyzing step includes
detecting amplicons comprising the structural rearrangements; and
reporting the presence of the tumor in the subject when the amplicons comprising the selected variant are detected.

7. The method of claim 3, wherein the analyzing step reports the structural rearrangements and genomic mutations.

8. The method of claim 3, wherein the structural rearrangement is selected for being likely to persist in the tumor.

9. The method of claim 1, further comprising partitioning the sample into partitions, and performing the exponential amplification and the detecting step in the partitions.

10. The method of claim 1, wherein the sample comprises blood or plasma from the subject and the nucleic acid includes cell-free DNA.

11. A method for variant detection, the method comprising the steps of:
providing a sample comprising one or more target nucleic acids;
performing an pre-amplification to preferentially increase abundance of copies of selected structural sequence rearrangements in the sample;
aliquoting the sample into a plurality of partitions;
conducting polymerase chain reaction (PCR) in the partitions; and
detecting amplicons in the partitions comprising at least one of the structural sequence rearrangements.

12. The method according to claim 11, wherein the pre-amplification step is selected from asymmetric incremental amplification and exponential amplification.

13. The method according to claim 12, wherein the asymmetric incremental amplification comprises the steps of:
providing a pair of primers capable of amplification of a target nucleic acid, wherein the pair of primers comprises a primer-H and a primer-L, wherein the melting temperature of primer-H is at least about 10° C. higher than the melting temperature of primer-L, and wherein primer-L comprises a sequence complementary to a fragment of the elongation product of primer-H; and
thermocycling the sample with an annealing temperature at which primer-H anneals but primer-L does not anneal.

14. The method of claim 13, wherein the PCR uses primer-H and primer-L in a thermocycle with an annealing temperature at which both primer-H and primer-L anneal.

15. The method of claim 11, wherein a portion of a reaction mixture of the pre-amplification is used as an input for the PCR.

16. The method of claim 15, wherein the portion of the reaction mixture is not subject to any clean-up step and the PCR is performed by adding components of a PCR reagent mixture to the portion of the reaction mixture.

17. The method of claim 11, further comprising determining quantities of the selected structural sequence rearrangements in the sample using (i) counts of the partitions in which amplicons are detected and (ii) a measure of the increase of the abundance of the copies of the selected structural sequence rearrangements yielded by the pre-amplification.

18. The method of claim 11, wherein the pre-amplification and the PCR are performed using the same set of primers.

19. The method of claim 11, wherein PCR reaction is digital PCR (dPCR).

20. The method of claim 11, wherein the aliquoting step includes diluting the sample so that each partition includes, on average, several template molecules for the PCR reaction, wherein the partitions include fluorescent hydrolysis probes.

21. The method of claim 11, wherein the PCR step includes multiplex dPCR.

22. The method of claim 11, wherein the detecting step detects one or more of single nucleotide variants (SNVs), insertions and deletions (indels), duplications, copy-number variants (CNVs), inversions, and translocations.

23. The method of claim 11, wherein the sample includes cell-free DNA from a cancer patient.

24. The method of claim 21, further comprising detection of the wildtype cfDNA sequence, wherein the method further comprises providing a pair of primers capable of binding to the wildtype cfDNA sequence and probes capable of binding to wildtype cfDNA sequence.
